(19) United States
(12) Patent Application Publication
(10) Pub. No.: US 2005/0079507 A1
(43) Pub. Date: Apr. 14, 2005

(54) Target evaluation using biological membrane arrays

(76) Inventor: Ye Fang, Painted Post, NY (US)

Correspondence Address: CORNING INCORPORATED, SP-TI-3-1, CORNING, NY, 14831, US

(21) Appl. No.: 10/682,712

(22) Filed: Oct. 9, 2003

(51) Int. Cl. C12Q 1/68; G01N 33/53; G01N 33/567

(52) U.S. Cl. 435/6; 435/7.2

(57) ABSTRACT

Novel uses of biological membrane microarrays and a new product platform or assembly are described. The invention involves cell membranes from different tissues or cells or organelles to fabricate tissue-specific cell membrane microarrays. The invention provides methods for identifying the relative distribution and/or abnormal expression levels of different membrane bound proteins, including G protein coupled receptors, in specific tissues or cells. In addition, the invention provides methods for screening target proteins that interact with membrane receptors.

### FIELD OF INVENTION

The invention relates to biological membrane arrays, particularly membrane-protein associated arrays. In particular, the invention pertains to the use of microarrays with tissue specific cell membranes for identifying and studying the distribution or abnormal levels of potential drug targets in certain tissues or cells. The invention also describes a kit and a method to screen and/or “fish-out” target proteins that interact with membrane-associated proteins and lipid receptors.

### BACKGROUND

Drug targets are mostly proteins that play a fundamental role in the on-set or progression of a particular disease. Until recently, pharmaceutical researchers have been limited to studying only approximately 500 biological targets (Drews, J., “Drug Discovery: A Historical Perspective” Science 2000, 287, 1960-1963). With the completion of the sequencing of the human genome, the number of available and potential biological targets is being expanded vastly. (International Human Genome Sequencing Consortium, “Initial Sequencing and Analysis of the Human Genome,” Nature 2001, 409, 860-921; J. Venter, et al., “The Sequence of the Human Genome,” Science 2001, 291 1304-1351.) Pharmaceutical and biotechnology companies are developing a large number of these newly identified potential targets for advancing the drug discovery process. Many other potential targets, however, have yet to be validated; meaning that their roles in causing disease are not completely understood.

The numbers of potential targets uncovered through genomics-based methods have created an enormous need for target evaluation technologies. Traditional drug discovery methods, however, have and can address only a limited number of target families. This situation suggests that the conventional methods have become “boxed in.” That is, the methods are unable to create as rapidly the numbers of novel drugs (e.g., three to five per year) that will be necessary to meet the business goals of the major pharmaceutical companies. The traditional methods are unlikely to provide breakthrough therapies for major diseases, such as cardiovascular diseases, neurodegenerative diseases, cancers, and type-2 diabetes, or other largely unmet medical needs. For these reasons, target evaluation has become one of the fastest growing and most critical fields of genomic research. Establishment of a stronger link between the target protein and the disease would lead to a lower failure rate when drugs proceed to clinical trials, and a shorter list of targets that have been proven to be valuable as drug targets would lead to greater success. In addition, a more rapid means of achieving better understanding of protein function would shorten the target evaluation process.

Target evaluation generally includes three major, critical stages: 1) target screening, 2) target identification, and 3) target validation. As the first and/or an early phase in target evaluation, the target screening stage involves identifying molecules that may be associated with a disease process (e.g., up-regulation of a particular gene identified through gene expression analysis). Target identification involves identifying molecules that clearly play a role in a disease process. As such, this type of approach provides a greater degree of certainty, but a possibility still exists that the identified targets will not be the best species or attach to the best binding sites to interfere with a disease process, or they may not be “druggable.” If all is successful, one may proceed to target validation, which is the process of determining which among the selected molecules leads to a phenotypic change when modulated, suggesting it may have value as a therapeutic target.

For evaluation purposes, nothing provides more a compelling validation for a target than membrane-bound proteins and other cell surface molecules, given that, to date from a historical point of view, membrane proteins have been most successful drug targets. For instance, G protein-coupled receptors (GPCRs), one subclass of membrane proteins, represent the single most important class of drug targets. Approximately 50% of current drugs target GPCRs; about 20% of the top 50 best selling drugs target GPCRs; more than $23.5 billion in annual pharmaceutical sales are ascribed to medications that address this target class. (Drews, J., “Drug Discovery: A Historical Perspective” Science 2000, 287, 1960-1963; Ma, P., and Zemmel, R., “Value of Novelty” Nat. Rev. Drug Discov. 2002, v.1, 571-572.) Ion channels and tyrosine kinase receptors, two other sub-families of membrane proteins, are also successful targets for modern drugs.

Applying the latest technologies to target evaluation is the first and crucial step in genomics-based drug disovery. Microarray technologies could enable a massively parallel approach to target identification. For instance, DNA microarray technologies have been used for gene expression profiling and single nucleotide polymorphism (SNP); and protein microarrays have been applied for protein expression profiling, and for protein-protein interaction studies. Together with proteomics, advanced chemical technologies (e.g., combinatorial chemistry, chemical genomics and chemogenomics), and high-throughput screening, genomics- and proteomics-based drug discovery has the potential to create drugs that can address large unmet medical needs. Robust and high-throughput methods of target identification and validation will be necessary to realize this potential, given that it is costly to sort through the targets one by one. Therefore, methods and the use of biological membrane microarrays including membrane protein microarrays (Fang, et al., “Membrane Protein Microarrays,” J. Am. Chem. Soc. 2002, 124, 2394-2395) for target evaluation should benefit drug discovery and development against one of the most important drug target classes.

### SUMMARY OF THE INVENTION

The present invention describes a kit or assembly, and methods that use biological membrane microarrays for target evaluation, which is an important phase in the drug discovery and development process. Generally, target evaluation employing biological membrane microarrays can be applied to a variety of purposes. These uses may include, but are not limited necessarily, to the following assays or categories of use: (1) determining the relative tissue distribution of a particular target; (2) determining the abnormal expression level of a particular target in a disease tissue or an abnormal cell; (3) determining protein-protein interactions; and (4) determining lipid receptor-protein interactions.

According to the invention, the kit can be used for biological, biochemical, or chemical analysis, and comprises a device and a reagent solution. The device includes a substrate having a functionalized surface for supporting a plurality of microspots of either biological membranes (e.g., cellular, lipid, natural or synthetic), membrane-bound proteins, or lipid receptors, arranged in an ordered fashion. The reagent solution may include a binder, marker, or a target protein. The device is characterized as suited for evaluating certain targets and performing any one of the above assays.

The method of using biological membrane microarrays for target evaluation comprises: (1) providing a microarray having a number of probe microspots deposited on a substrate surface; (2) applying a solution containing a binder, marker, or a protein to said microarray; and (3) performing an assay for one of the aforementioned uses.

In a first aspect, a method for determining tissue distribution of a particular drug target may comprise: 1) providing a microarray having a number of probe microspots of cell membranes from different tissues or cells; 2) providing a solution containing a labeled or unlabeled binder or marker, in which the binder or marker can specifically bind to the drug target in the probe microspot; 3) applying the solution to the microarray; and 4) determining the level of drug target in each different probe microspot.

In a second aspect, a method for determinating abnormal expression level of a particular drug target in a disease tissue or an abnormal cell may comprise: 1) providing a microarray having a number of probe microspots of cell membranes, in which said cell membranes are from both a normal tissue cell and an analogous diseased- or abnormal tissue cell; 2) providing a solution containing a labeled or unlabeled binder or marker, in which the binder or marker can specifically bind to the drug target in the probe microspot; 3) applying the solution to the microarray; and 4) comparing the level of the drug target in the disease-tissue cell with that in said normal tissue cell. The sample of diseased or abnormal tissue cells can represent a variety of stages over the course of progression of a disease. That is, a number of microspots each can contain tissue or cell samples from either an initial or on-set stage, an intermediate or later stage, or terminal stage.

In a third aspect, a method for determining protein-protein interaction may comprise: 1) providing a microarray of probe protein receptors embedded in lipid membranes; 2) providing a solution containing a target protein which is either labeled or unlabeled; 3) applying the solution to the microarray; and 4) determining the binding profiles of the target protein to the probe receptor in the microarrays.

In a fourth aspect, a method for determining lipid receptor-protein interaction may comprise: 1) providing a microarray of probe lipid receptors, which are either purified or embedded with lipid membranes; 2) providing a solution containing a target protein, which is either labeled or unlabeled; 3) applying the solution to the microarray; and 4) determining the binding profiles of the target protein to the lipid receptors in said microarray. The lipid receptor can be a ganglioside, a phosphatidylinositol phosphate (PIP), a sphingolipid, cholesterol, or a lipid-raft domain.

In another aspect, a method to normalize signals due to different expression levels of a particular drug target in a tissue or cell membrane comprises: 1) providing cell membrane preparations from different tissue cells, either normal or abnormal; 2) reformulating the cell membrane preparations in a buffer containing pH buffer, inorganic salt, BSA and sucrose, optionally glycerol, such that the total membrane protein concentration is identical or same for said membrane preparations; and 3) depositing the cell membrane preparations onto a substrate surface to form a microarray. Optionally, one may incorporate a homogenization step after the reformulating the cell membranes, before depositing onto the substrate.

Additional features and advantages of the present invention will be revealed in the following detailed description. Both the foregoing summary and the following detailed description and examples are merely representative of the invention, and are intended to provide an overview for understanding the invention as claimed.

### BRIEF DESCRIPTION OF THE FIGURES

FIG. 1 is a schematic illustration representing a microarray of cell membranes derived from different tissue cells. Each microspot (10) on the surface (12) of the microarray represents a cell membrane from a specific type of tissue or cells. When a labeled or unlabeled binder or marker for a particular membrane-bound protein binds to the cell membrane microspots, the relative binding signal among different microspots reflects the relative distribution or expression label of the particular membrane proteins within the different tissues or cells.

FIG. 2 is a schematic illustration showing a microarray of cell membranes derived from normal tissue cells and abnormal tissue cells. Two sets of cell membranes are included in the same microarray. One set includes normal tissues or cells, while the second set includes abnormal or diseased (e.g., tumor, cancer) tissue or cell counterparts. Using the normal counterparts are used as a baseline reference, when a labeled or unlabeled binder or marker for a particular membrane-bound protein binds to the cell membrane microspots, the difference or relative intensity of binding signals between the normal and abnormal counterparts indicates that particular proteins may be either up- or down-regulated in the abnormal tissues or cells.

FIG. 3 is a false-color fluorescence image of a microarray having three different cell membranes from CHO (A), HEK-293 (B), and A341(C) cells. The image is taken after the microarry has been assayed using a binding solution containing 4 nM of TMR-epidermal growth factor (EGF). The total binding signal of A341 cell membrane microspots in the array is about 4-6 fold higher than that of either the CHO or HEK-293 cell membrane microspots. This result confirms the fact that the EGF receptor is highly expressed (˜10⁶ copies per cell) in the tumor A341 cells, but is expressed at a relatively low level in CHO or HEK-293 cells, since EGF is a natural ligand for the EGF receptor.

### DETAILED DESCRIPTION OF THE INVENTION

### Section I—Definitions

Before describing the present invention in detail, this invention is not necessarily limited to specific compositions, reagents, process steps, or equipment, as such may vary. As used in this specification and the appended claims, the singular forms “a,” “an,” and “the” include plural referents unless the context clearly dictates otherwise. It is also to be understood that the terminology used herein is for the purpose of describing particular embodiments only, and is not intended to be limiting. All technical and scientific terms used herein have the usual meaning conventionally understood by persons skilled in the art to which this invention pertains, unless context defines otherwise.

The term “binder,” or “marker” refers to a biological, chemical, or biochemical molecule that can recognize and bind with a particular membrane protein. The binder or marker can be a ligand, a protein, an antibody, or an aptamer (e.g., DNA-, RNA-, or peptide-aptamers). When the binder is a protein, an antibody that can bind with the protein may be used as a readout molecule, preferably, in a secondary or sequential step. The binder or marker can be either labeled or unlabeled. If labeled, the label can be any of the following: a fluorescent tag, a radio-isotope, a nano-particle (e.g., gold particle, quantum dots, etc.), or biotin.

The term “functionalization” as used herein relates to modification of a solid substrate to provide a plurality of functional groups on the substrate surface. The phrase “functionalized surface” as used herein refers to a substrate surface that has been modified to have a plurality of functional groups present thereon. The surface may have an amine-presenting functionality (e.g., γ-amino-propylsilane (GAPS) coating), or may be coated with amine presenting polymers such as chitosan and poly(ethyleneimine).

The term “ink,” “medium,” or “composition” refers to a buffered medium or aqueous solution containing components or reagents that can stabilize a biological membrane either in solution or after deposition onto a substrate, and/or improve the consistency or reproducibility of the amount of membrane-containing solution transferred from a disposition device to the substrate. The components include a combination of six classes of reagents: 1) a pH buffer reagent; 2) a monovalent or divalent, inorganic salt; 3) a membrane stabilizer; 4) a solution viscosity control reagent; 5) a water-soluble protein; and/or, 6) a protease inhibitor. In some embodiments, a mixture of at least two of the six classes of components with biological membranes may be present together in solution.

The term “microspot” refers to a discrete or defined area, locus, or spot on the surface of a substrate, containing a biological probe. The term “receptor microspot” refers to a microspot containing a deposit of biological membrane presenting binding functional moieties or molecules, such a ganglioside, phosphatidylinositol phosphate (PIP), sphingolipid, or membrane-proteins. The membrane-protein may include a GPCR, a ligand-gated ion channel receptor, a tyrosine kinase receptor, a serine/threonine kinase receptor, an immune receptor, or a guanylate cyclase receptor.

The term “probe” or “probe receptor” refers to a cell membrane, a cell membrane fragment, a receptor molecule bound in the cell membrane (e.g., GPCR, tyrosine kinase receptor, ion channel), which according to the nomenclature recommended by B. Phimister (Nature Genetics 1999, 21 supplement, pp. 1-60.), is immobilized to a substrate surface. Preferably, probes are arranged in a spatially addressable manner to form an array of microspots. When the array is exposed to a sample of interest, molecules in the sample selectively and specifically binds to their binding partners (i.e., probes). The binding of a “target” to the probes occurs to an extent determined by the concentration of that “target” molecule and its affinity for a particular probe.

The term “substrate” or “substrate surface” as used herein refers to a solid or semi-solid, or porous material (e.g., micro- or nano-scale pores), which can form a stable support. The substrate surface can be selected from a variety of materials.

### Section II—Detailed Description

Drug discovery and development is the process of creating and evaluating drugs for the safe and effective treatment of human disease. This process generally comprises a number of steps: target evaluation (target screening, target identification, and target validation), drug discovery (structural biology, lead generation, lead optimization and process research and development), lead identification and validation, preclinical development, and clinical development.

Critical information needs to be collected before a protein could be classified as a “druggable” target. (Hopkins, A. L., and Groom, C. R., “The Druggable Genome,” Nat. Rev. Drug Discov. 2002, v. 1, 727-730.) Such information relates to the proteins' particular gene sequence or sequence homology, differential gene expression data, differential protein-expression data, protein structure, genetic networks or protein pathways, protein-protein interactions, gene functions, protein functions, molecular pathology, physiological and pathological roles in model-organism-based systems. The model-organism-based systems include disease models based on yeast or on invertebrates, non-human mammalian disease models, knockout-mouse, transgenic mouse, and human tissues including stem cells.

Given that membrane proteins have been most successful drug targets to date, membrane-bound proteins and other cell surface molecules provide attractive means to evaluate or validate a target. Membrane-bound proteins represent the single most important class of drug targets. Approximately 50% of the current drug targets are membrane bound; 20% of the top 200 best selling drugs target G protein-coupled receptors (GPCRs). The main reasons lie in the critical roles cell membranes and their associated molecules play in cells. Cell membranes play extremely important roles in maintaining the integrity of living cells. Cell membranes regulate the transport of molecules, contain molecules responsible for cell adhesion in the formation of tissues, control information flow between cells, generate signals to alter cell behavior, as well as can separate molecules for cell signaling and energy generation. In addition, cell membranes also involve in the recognition and sequential infection of toxins, bacteria, and virus.

Since analysis of protein expression from mRNA levels using DNA microarrays is prone to artifacts and does not provide information regarding post-translational modifications; and proteins are the molecular entities that bind drugs, the analysis of protein expression level directly and protein-protein interaction provide direct information about a potential drug target. By profiling the differential expression of proteins using antibody arrays and correlating those changes to a disease phenotype (Mitchell, P. “A Perspective on Protein Microarrays,” Nat. Biotechnol. 2002, 20, 225-229; MacBeath, G. and Schreber, S. L. “Printing Proteins as Microarrays for High-Throughput Function Determination,” Science 2000, 289, 1760-1763; Schweitzer, B. et al. “Immunoassays with Rolling Circle DNA Amplification: A Versatile Platform for Ultrasensitive Antigen Detection,” Proc. Natl. Acad. Sci. USA 2000, 97, 10113-10119), several putative targets (and biomarkers) to a particular disease may be identified. Other “bait” molecules include peptides, aptamers, and carbohydrates.

Previously, we have demonstrated that one may fabricate biological membrane microarrays using conventional robotic pin printing technologies and biological membranes including cell membrane preparations containing GPCRs from a cell line over-expressing the receptor. (U.S. patent application Ser. No. 09/974,415 (U.S. Patent Publication No. 2002/0019015 A1), and Ser. No. 09/854,786, (U.S. Patent Publication No. 2002/0094544 A1) the contents of which are incorporated herein by reference). The biological membranes may take the form of either a supported lipid bilayer membrane, a bilayer vesicle, a lipid micelle, at least a partially free-suspended lipid membrane, or a lipid membrane in a nano-channel of a substrate, with or without embedded membrane-proteins. The membrane-protein is a GPCR, a ligand-gated ion channel receptor, a tyrosine kinase receptor, a serine/threonine kinase receptor, an immune receptor, or a guanylate cyclase receptor.

These kinds of arrays can be prepared under ambient conditions, stored at about 4° C., and still retain their functionality for an extended period of time thereafter. These kinds of arrays have been used for a number of applications. For instance, GPCR arrays can be used for pharmacologically profiling of drug compounds or screening for compounds that bind to a GPCR (Fang, Y. et al. (2002) “Membrane Protein Microarrays.” J. Am. Chem. Soc. 124, 2394-2395; Fang, Y. et al. (2003) “G Protein-Coupled Receptor Microarrays for Drug Discovery,” Drug Discov. Today, 8, 755-761). Microarrays containing gangliosides have been used for detecting toxins in a sample and screening toxin inhibitiors (Fang, Y. et al. “Ganglioside Microarrays for Toxin Detection,” Langmiur, 2003, 19, 1500-1505); microarrays of lipid receptors such as phosphatidylinositol phosphate (PIP) can be used for identifying proteins that interact with these lipid molecules (U.S. patent application Ser. No. 10/392,193).

The present invention extends the usage of biological membrane microarrays for determining expression profiles of a membrane-bound protein in different tissue cells. The invention can be applied to uncover the abnormal level of a membrane-bound protein in disease tissue cells or abnormal cells, or study the interaction of a membrane-bound protein or a lipid receptor with other proteins in their native environment. Similar to GPCR arrays, cell membrane microarrays can be fabricated with pin-printing technology. (See for example, U.S. Patent Application Publication No. 2002/0019015 A1.)

I. Expression Level Analysis of a Membrane-Bound Protein in Different Tissue Cells

Distribution analysis (i.e., expression level analysis) of a target protein in different tissues including cancer or tumor cells is very important for understanding the biological and/or physiological functions of the target protein. GPCRs and any other membrane proteins are distinctly expressed in different types of cells or tissues. Examples may include: (1) Tachykinin NK receptors and angiotensin receptors are highly expressed in central neuron systems (A. Saria, “The Tachyknin NK1 Receptor in the Brian: Pharmacology and Putative Functions,” European J. Pharmacology, 1999, 375, 51-601); (2) Neuropeptide Y receptors are highly expressed in brain, but not or significantly lower expressed in several peripheral tissues including heart, spleen, lung, liver, skeletal muscle and kidney (A. Inui, “Neuropeptide Y Feeding Receptors: Are Multiple Subtypes Involved?” Trends in Pharmacological Sciences (TiPS) 1999, 20, 43-46); (3) Galanin receptors subtype 1 is highly expressed in brain and small intestinal tissue, in Bowes melanoma cells, in gastrointestinal tract from the oseophagus to the rectum. However, the GAL subtype 2 is widely distributed in several central and peripheral tissues; (4) CXC chemokine receptor-4 is diffusely and homogeneously expressed in 59 cancers, which were further divided into 28 high-expression and 31 low-expression cancers (Kato, M. et al. “Expression Pattern of CXC Chemokine Receptor-4 is Correlated with Lymph Node Metastasis in Human Invasive Ductal Carcinoma”, Breast Cancer Res. 2003, 5, R144-R150). High-CXCR4 tumors showed more extensive nodal metastasis in comparison with low-expression tumors. Also, some GPCRs or their mutants are distinctly expressed in different cancer or tumor cells.

Other possible species of membrane-bound proteins may be selected from groups. For example, Met tyrosine kinase receptor is expressed at a significantly high level in bone metastases (Knudsen, B. S. et al. “High-Expression of the Met Receptor in Prostate Cancer Metastasis to Bone”, Urology. 2002, 60,1113-1117). Also, epidermal growth factor receptor (EGFR) is commonly overexpressed in adult high-grade gliomas. About 40˜50% of such tumors demonstrate amplification of the EGFR gene, often with rearrangement and constitutive activation of the gene product. This results suggest that EGFR might play a role in the malignant progression of a subset of these neoplasms (Bredel, M. et al. “Epidermal Growth Factor Receptor Expression and Gene Amplification in High-Grade Non-Brainstem Gliomas of Childhood,” Clinical Cancer Research 1999, 5, 1786-1792).

Traditional methods used for studying the distribution of a particular target protein, including membrane-bound proteins, in different tissues can be classified into two major types. The first type is based on mRNA level analysis, such as (1) Northern blot analysis; (2) RNase protection method; and (3) reverse transcriptase PCR methods; and (4) in-situ hybridization histochemistry. The second type uses radio-labeled ligands and/or antibodies to map the distribution of particular receptors (so-called in-vitro autoradiography). Normally, these two types of applications give rise to similar distribution profiles for a given receptor. Unfortunately, however, these methods are not high-throughput and sometime do not produce comparable results. This problem arises because, first, protein expression analysis based on mRNA levels is prone to generate artifacts and does not provide information regarding post-translation modifications; and, second, the sensitivity of in vitro autoradiography is relatively low.

The present invention addresses this and other issues associated with the limitations and poor predictability of animal-based strategies. When integrated with microarray technology, the use of human tissues and cells (e.g., stem cell) early in the discovery process could produce the breakthrough advances or synergies that significantly accelerate and improve the efficiency of the overall drug discovery process. That is, for example, target identification and validation, safety testing, compound selection and crucial decision making on parameters to advance products to clinical testing. Human tissues have become used widely throughout the drug discovery and development processes for drugs that target human subjects.

The particular embodiments of the invention are described in terms of tissue-specific cell membranes. Cell membranes can be prepared from different normal or disease-related tissues or cells by using state-of-the-art approaches. For instance, sub-cellular fractionation techniques can partially separate and purify several important biological membranes, including the plasma and mitochondrial membranes, from many kinds of cells. Such biological membrane preparations generally contain natural or native compositions of membrane associated components (e.g., receptors, lipids, or in some cases intracellular proteins that bind to receptors or membranes). Cell membranes are assemblies of membrane-proteins, carbohydrates, and lipids held together by non-covalent forces. Membrane proteins determine the functionality of cell membranes, serving as pumps, gates, receptors, cell adhesion molecules, energy transducers, and enzymes. Peripheral membrane proteins are associated with the surfaces of membranes, while integral membrane proteins are embedded in the membrane and may pass through the lipid bilayer one or more times.

The present invention pertains to a method for determining tissue distribution of a particular drug target, the method comprises: 1) providing a microarray having a number of microspots of cell membranes from different tissues or cells; 2) providing a solution containing either a labeled or unlabeled binder or marker, in which said marker can specifically bind to said drug target in said microspot; 3) applying said solution to said microarray; and 4) determine the level of said drug target in different microspots.

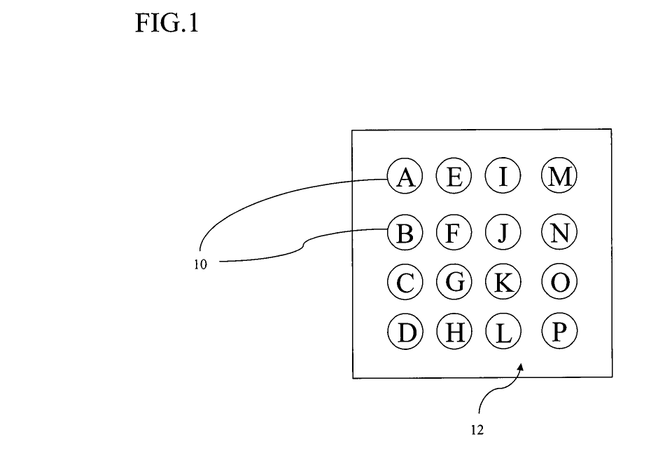

FIG. 1 shows a schematic of a microarray of cell membranes derived from different types of tissues or cells. A cell membrane from a specific type of tissue or cells is contained within at least one microspot (10) on the surface (12) of the microarray. Replicates containing the same sample, preferably, are included for reliable statistical analysis of each assay. In one embodiment, the cell membrane fragments are immobilized randomly immobilized onto a substrate surface. In another preferred embodiment, the cell membrane fragments are immobilized onto the substrate surface in a pre-determined orientation (i.e., either the intracellular side facing the substrate surface, or the extracellular side facing the substrate surface). The orientation of cell membranes in the array may provide better assay sensitivity, since a particular binder or marker can more easily interact with the target membrane protein from one side of the cell membranes. All integral proteins bind asymmetrically to the lipid bilayer; each type of integral membrane protein has a single, specific orientation with respect to the cytosolic and exoplasmic faces of a cellular membrane. This absolute asymmetry in protein orientation generates the different characteristics associated with the two faces of a membrane. In again another embodiment, the cell membranes are immobilized onto a substrate surface having a nanoporous sub-structure such that the membranes are at least partially and freely suspended across the nano-scale pores (Hennesthal, C. and Steinem, C. “Pore-Spanning Lipid Bilayers Visualized by Scanning Force Microscopy,” J. Am. Chem. Soc. 2000; 122, 8085-8086). This type of immobilization might allow both sides of membranes being accessible to the binder or marker.

When a labeled or unlabeled binder or marker for a particular membrane-bound protein binds to the cell membrane microspots, the relative binding signal among different microspots reflects the relative distribution or expression label of the particular membrane proteins within the different tissues or cells. The binder or marker refers to a biological or biochemical molecule that can specifically bind to said drug target in said probe microspot. The binder or marker is a ligand, an antibody, a protein, or an aptamer. The aptamer could be, for example, a DNA/RNA aptamer, or a peptide aptamer. The binder or marker can be either unlabeled or labeled by a fluorescent tag, a radio-isotope, a nano-particle, or biotin. When an unlabeled protein is used as a marker, a labeled antibody, which can bind with said unlabeled protein, might be used to function as a readout; the labeled antibody can be applied to the microarrays in a sequential step (similar to the so-called “Sandwich” assays developed for antibody microarrays).

The present invention has advantages over traditional methods for protein expression level profiling. For example, the present method can not only provide information about the relative expression level of a particular protein in a much larger set of tissues or cells within a single assay, and also retain the location information of the protein since only cell membrane-associated proteins are arrayed and analyzed.

II. Identification of a Membrane-Bound Target Associated with a Disease Tissue Cell.

To understand the molecular pathology of the onset or progression of a disease, including cancers and tumors, nothing is more important than identifying the abnormal expression level of a particular target protein in a disease tissue or an abnormal cell. This is because in many cases, over expression of some specific gene products, such as epidermal growth factor receptor (EGFR) and insulin-like growth factor receptors, have been linked as a causative factor to certain kinds of cancers (e.g., V. T. DeVita, S. Hellman, S. A. Rosenberg, Eds “Cancer: Principles and Practice of Oncology”, Lippincott-Raven, Philadelphia, 1997). This kind of analysis is one of the most important studies to tie a biological molecule or target to a pathogenesis or diseases in later stages for target evaluation (i.e., target identification and validation). Many molecular tools are available for target validation, including antisense oligonucleotides, ribozymes, dominant negative mutants, neutralizing antibodies, and mouse transgenics/knockouts. Often multiple approaches must be evaluated.

The present invention provides a method to determinate abnormal expression level of a particular drug target in a disease tissue or an abnormal cell. The method comprises: 1) providing a microarray having a number of microspots of cell membranes, in which said cell membranes are from both a normal tissue cell and an analogous diseased- or abnormal tissue cell; 2) providing a solution containing either a labeled or unlabeled binder or marker, in which the marker can specifically bind to said drug target in said probe microspot; 3) applying said solution to said microarray; and 4) comparing the level of said drug target in said disease-tissue cell with that in said normal tissue cell.

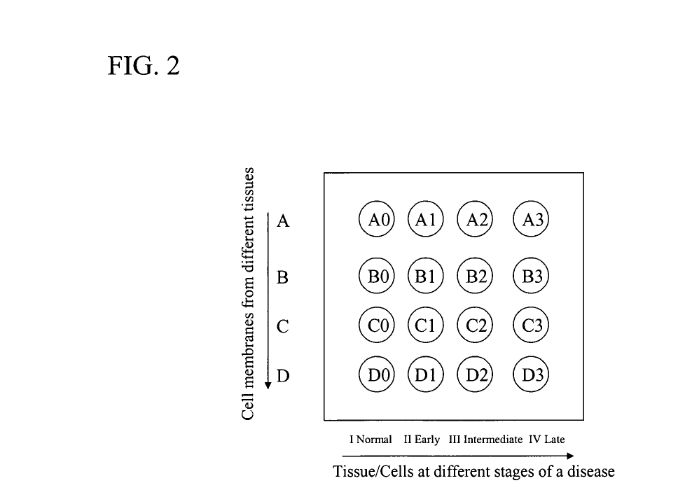

FIG. 2 represents a schematic of a microarray of cell membranes in microspots from derived from both two sets. As indicated along the horizontal axis, one set includes normal tissues or cells (I), while the second set includes abnormal or diseased (e.g., tumor, cancer) tissue or cell counterparts. The samples derived from the abnormal tissue or cells can be further grouped into an initial or on-set stage (II), an intermediate or later stage (III), or terminal stage (IV). The normal counterparts are used as baseline references. Different type of tissue or cellular specimenscan be arranged in sequence, such as indicated on the vertical axis. When a labeled or unlabeled binder or marker for a particular membrane-bound protein binds to the cell membrane microspots, the difference or relative intensity of binding signals between the normal and abnormal counterparts indicates that particular proteins may be either up- or down-regulated in the abnormal tissues or cells.

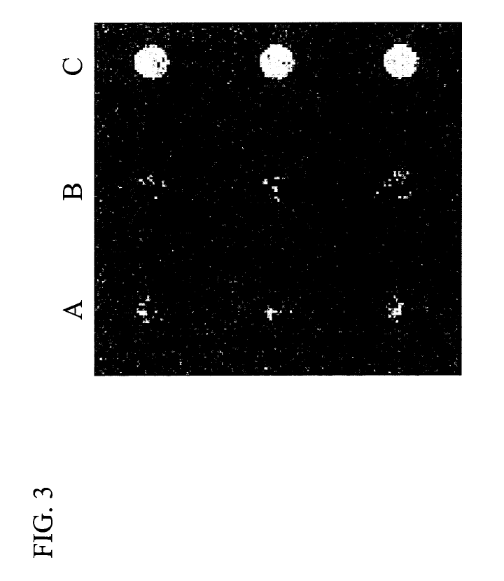

FIG. 3 shows a false-color fluorescence image of a microarray having three different cell membranes from CHO (A), HEK-293 (B), and A341(C) cells. The image is taken after the microarray is assayed using a binding solution containing 4 nM of TMR-epidermal growth factor (EGF). The total binding signal of A341 cell membrane microspots in the array is about 4-6 fold higher than that of either the CHO or HEK-293 cell membrane microspots. The difference in binding of TMR-EGF could be even more significant if one subtracts the background signal, which is mainly due to the intrinsic auto-fluorescence of the cell membranes, and the non-specific binding signal of the labeled EGF to the cell membranes. A saturation assay may be a more preferred and better way to examine the amount of active receptors in each cell membrane microspot. Results obtained using saturation assays show that the amount of active EGFRs in A341 cell membrane microspots is about 100˜500 fold higher than that in both CHO or HEK cells (data not shown). These results confirm the fact that the EGF receptor is highly expressed (˜10⁵-10⁶ copies per cell) in the tumor A341 cells, but not in CHO or HEK-293 cells (˜tens or hundreds of copies per cell), since EGF is a natural ligand for the EGF receptor.

III. Protein-Protein Interaction Using Membrane Protein Microarrays.

Cell-surface molecules experience extensive interaction with intracellular proteins. For example, agonist-binding G protein-coupled receptors (GPCRs) interact and activate heterotrimeric G proteins, which then regulate the activity of specific cellular effectors. Beyond the G protein paradigm, GPCRs can interact with members of diverse families of intracellular proteins. Among these proteins may include, for instance, polyproline-binding proteins such as those containing Sh3 domains, arresting, G protein-coupled receptor kinases (GRK), small GTP-binding proteins, SH2 domain-containing proteins, or PDZ domain-containing proteins. Membrane domains containing phosphatidyllinositol phosphate (PIP) are targets for many pleckstrin homology (PH)-containing proteins such as PLC-β and ARF protein exchange factor GRP1.

Knowledge of the cellular signaling pathways can be helpful for exploiting rational targets that prove to be druggable. A cell-based assays to study protein-protein interactions, however, may fail because in some situations, such as with tumor suppressor genes (e.g., Ras), a protein target is no longer present in the tumor. For example, many of the early approaches to inhibit Ras protein function failed in Ras suppressed tumor cells. In contrast to conventional cell-based techniques, the present invention offers not only high-throughout, larger-scale parallel analysis of protein-protein interactions, but also a method for profile membrane-interacting proteins within cells, and even damaged or gene-suppressed, pathogenic cells.

The invention discloses a method for determining protein-protein interaction, which may comprise: 1) providing a microarray of probe protein receptors embedded in lipid membranes; 2) providing a solution containing a target protein which is either labeled or unlabeled; 3) applying the solution to the microarray; and 4) determining the binding profiles of the target protein to the probe receptor in the microarrays. In an alternative embodiment or application, the present method can be used to determine or profile the level of membrane-interacting protein expression within a cell. The method comprises: 1) providing a microarray of probe protein receptors embedded in lipid membranes; 2) providing a solution of cell lysates containing a target protein, which can be either a natural or a fusion protein (e.g., GFP, YFP, or His-tag); 3) applying the solution to the microarray; and 4) determining the binding profiles of the target protein to the probe receptor in the microarrays.

The probe-receptors can be a membrane-protein including a GPCR, a ligand-gated ion channel receptor, a tyrosine kinase receptor, a serine/threonine kinase receptor, an immune receptor, or a guanylate cyclase receptor. The probe-repectors are associated with a biological membrane, which may take the form of either a supported lipid bilayer membrane, a bilayer vesicle, a lipid micelle, at least a partially free-suspended lipid membrane, or a lipid membrane in a nano-channel of a substrate, with or without embedded membrane-proteins. The membrane-proteins, preferably, are in a purified state, and reconstituted with a biological membrane.

IV. Lipid Receptor-Protein Interaction Using Lipid Receptor Microarrays.

Another class of cell membrane-associated molecules are carbohydrates covalently linked to proteins (glycoproteins) or lipids (glycolipids). Glycolipid molecules have a phospholipid structure, which is embedded within the cell membrane, and at least one carbohydrate chain extending from the cell surface. The carbohydrate groups provide part of the structure that enables the glycolipid and glycoprotein molecules to perform recognition, reception and adhesion functions. In a plasma membrane, all of the oligosaccharides in glycolipids are on the exoplasmic surface. In addition, cholesterol and its derivatives constitute another important class of membrane lipids, the steroids.

Cholesterol regulates membrane fluidity and is a part of membrane signaling systems. For instance, bacterial toxins (e.g., from the genera Streptococcus, Bacillus, Clostridium, and Listeria) target cholesterol molecules. Hence, glycolipids and cholesterol molecules can be the target for toxin binding and sequential infection. A large number of bacterial toxins target carbohydrate-derivatized lipids on the cell surface, often with high specificity. These lipids, glycosylated derivatives of ceramides, referred to as sphingoglycolipids, can be classified into cerebrocides (ceramide monosaccharide), sulfatides (ceramide monosaccharide sulfates), and gangliosides (ceramide aoligosaccharides).

According to the present invention, a method for determining lipid receptor-protein interaction may comprise: 1) providing a microarray of lipid receptors, which are either purified or embedded with lipid membranes; 2) providing a solution containing a target protein, labeled or unlabeled; 3) applying said solution to said microarray; and 4) determining the binding profiles of said target protein with said lipid receptors in said microarray. Similar to the method for profiling protein-protein interactions, using protein receptors in Section III, above, an alternative embodiment or application of the present method can be used to determine or profile the level of membrane-interacting protein expression within a cell. The alternate method comprises: 1) providing a microarray of probe lipid receptors embedded in lipid membranes; 2) providing a solution of cell lysates containing a target protein, which can be either a natural or a fision protein (e.g., GFP, YFP, or His-tag); 3) applying the solution to the microarray; and 4) determining the binding profiles of the target protein to the probe receptor in the microarrays. As mentioned previously, the lipid receptor can be, but not necessarily limited to, a ganglioside, a phosphatidylinositol phosphate (PIP), a sphingolipid, cholesterol, or a lipid-raft domain.

The present invention can extend the applicable reach of the methods and use of microarrays such as described in U.S. patent application Ser. No. 10/602,242, or U.S. patent application Ser. No. 10/392,193, the contents of which are incorporated herein by reference. U.S. patent application Ser. No. 10/602,242, discloses methods and a device for toxin detection using ganglioside microarrays, while U.S. patent application Ser. No. 10/392,193, describes a universal readout assay to detect toxin using ganlioside microarrays, to detect PIP-binding protein using phosphoinositol (PIP) microarray, and to identify lipid rafts binding proteins using sphingolipid microarray.

V. Methods to Normalize Cell Membrane Preparations.

Sub-cellular fractionation techniques can partially separate and purify several important biological membranes, including the plasma and mitochondrial membranes, from many kinds of cells. Such biological membrane preparations generally have a varied distribution of lipid membrane fragments in different sizes and different concentrations of total membrane bound proteins. Therefore, a method to normalize the cell membranes is required for target screening and identification.

On the other hand, membrane preparation homogeneity is another important parameter, which can affect the analysis results. Homogeneity influences the packing density and uniformity of membrane fragments within a microspot, as well as the reproducibility of printing. Smaller and more homogeneous membrane fragments yield membrane microspots with better packing density and uniformity, as well as improved printing reproducibility; therefore, lead to more accurate and precise estimation of expression levels of a particular membrane-bound protein in different tissue cell membranes.

Normalization and homogeneity of the cell membrane preparations is one of several factors for success according to this present invention. One simple way is to use different cell membranes that are suspended in same buffer composition and contain the same amount of total membrane proteins. According to the invention, a method comprises: 1) providing cell membrane preparations from different tissue cells, either normal or abnormal; 2) reformulating the cell membrane preparations in a buffer containing pH buffer, inorganic salt, BSA and sucrose, optionally glycerol, such that the total membrane protein concentration is identical or same for said membrane preparations; and 3) depositing the cell membrane preparations onto a substrate surface to form a microarray. Optionally, one may incorporate a homogenization step after the reformulating the cell membranes, before depositing onto the substrate. The homogenization process can use, for example, either a Dounce homogenizer or a sonication device to break-down the membrane fragments to have a smaller size and more uniform distribution.

The present invention has been described both in general and in detail by way of examples. Persons skilled in the art will understand that the invention is not limited necessarily to the specific embodiments disclosed. Modifications and variations may be made without departing from the scope of the invention as defined by the following claims or their equivalents, including equivalent components presently known, or to be developed, which may be used within the scope of the present invention. Hence, unless changes otherwise depart from the scope of the invention, the changes should be construed as being included herein.

1. A method of using a biological membrane microarray for target evaluation, the method comprises: (1) providing a microarray having a number of probe microspots deposited on a substrate surface; (2) applying a solution containing a binder, marker, or a protein to said microarray; and (3) performing an assay for one of the following purposes: a) determining the relative tissue distribution of a particular target in different tissues or cells; b) determining the abnormal expression level of a particular target in a disease or abnormal tissue or cell; c) determining protein-protein interactions; or d) determining lipid receptor-protein interactions.

2. The method according to claim 1, wherein the method may include providing a formulation of cell membranes from a variety of tissues or cells to fabricate said microarray.

3. The method according to claim 1, wherein for either determining tissue distribution of a particular drug target or determining the abnormal expression level of a particular target in a disease or abnormal tissue or cell, said solution contains either a labeled or unlabeled binder or marker, in which said binder or marker can specifically bind to said drug target in said probe microspot.

4. The method according to claim 1, wherein said microarray has a number of probe microspots of cell membranes from different tissues or cells, for determining the relative level of said drug target in different probe microspots.

5. The method according to claim 1, wherein said microarray has a number of probe microspots of cell membranes from both a normal tissue cell and an analogous diseased or abnormal tissue cell, for comparing the relative level of said drug target in said diseased-tissue cell with the level of said drug target in said normal-tissue cell.

6. The method according to claim 3, wherein said binder or marker is a ligand, an antibody, a protein, or an aptamer.

7. The method according to claim 6, wherein when said binder or marker is an unlabeled protein, an antibody, which can bind with said unlabeled protein, functions as a readout.

8. The method according to claim 3, wherein said binder is labeled and said label is a fluorescent tag, a radio-isotope, a nano-particle, or biotin.

9. The method according to claim 1, wherein for either determining protein-protein interaction or determining lipid receptor-protein interaction, said solution contains a target protein, which is either labeled or unlabeled.

10. The method according to claim 1, wherein for either determining the expression profile of a target membrane-interacting protein in a cell sample, said solution contains a cell lysate.

11. The method according to claim 1, wherein said microarray has a number of probe protein receptors embedded in lipid membranes for determining the binding profiles of said target protein with said probe receptors.

12. The method according to claim 11, wherein said protein receptor is at least one of the following: a GPCR, a ligand-gated ion channel receptor, a tyrosine kinase receptor, a serine/threonine kinase receptor, an immune receptor, or a guanylate cyclase receptor.

13. The method according to claim 1, wherein said microarray has a number of probe lipid receptors that are either purified or embedded with lipid membranes, for determining the binding profiles of said target protein with said lipid receptors.

14. The method according to claim 13, wherein said lipid receptor can be a ganglioside, a phosphatidylinositol phosphate (PIP), a sphingolipid, cholesterol, or a lipid-raft domain.

15. A method for determining tissue distribution of a particular drug target, the method comprises: 1) providing a microarray having a number of microspots of cell membranes from different tissues or cells; 2) providing a solution containing either a labeled or unlabeled binder or marker, in which said marker can specifically bind to said drug target in said microspot; 3) applying said solution to said microarray; and 4) determine the level of said drug target in different microspots.

16. A method for determinating abnormal expression level of a particular drug target in a disease tissue or an abnormal cell, the method comprises: 1) providing a microarray having a number of microspots of cell membranes, in which said cell membranes are from both a normal tissue cell and an analogous diseased—or abnormal tissue cell; 2) providing a solution containing either a labeled or unlabeled binder or marker, in which the marker can specifically bind to said drug target in said probe microspot; 3) applying said solution to said microarray; and 4) comparing the level of said drug target in said disease-tissue cell with that in said normal tissue cell.

17. A method for determining protein-protein interaction, the method comprises: 1) providing a microarray of protein receptors embedded in lipid membranes; 2) providing a solution containing a target protein, which is ether labeled or unlabeled; 3) applying said solution to said microarray; and 4) determining the binding profiles of said target protein with said probe receptor in said microarrays.

18. A method for determining or profiling the relative level of membrane-interacting protein expression within a cell, the method comprises: 1) providing a microarray of probe protein receptors embedded in lipid membranes; 2) providing a solution of cell lysates containing a target protein, which can be either a natural or a fusion protein; 3) applying the solution to the microarray; and 4) determining the binding profiles of the target protein to the probe receptor in the microarrays.

19. A method for determining lipid receptor-protein interaction, the method comprises: 1) providing a microarray of lipid receptors, which are either purified or embedded with lipid membranes; 2) providing a solution containing a target protein, labeled or unlabeled; 3) applying said solution to said microarray; and 4) determining the binding profiles of said target protein with said lipid receptors in said microarray.

20. A method for determining or profiling the level of membrane-interacting protein expression within a cell, the method comprises: 1) providing a microarray of probe lipid receptors embedded in lipid membranes; 2) providing a solution of cell lysates containing a target protein, which can be either a natural or a fusion protein; 3) applying the solution to the microarray; and 4) determining the binding profiles of the target protein to the probe receptor in the microarrays.

21. A method to normalize signals due to different expression levels of a particular drug target in a tissue or cell membrane, the method comprises: 1) providing cell membrane preparations from different tissue cells, either normal or abnormal; 2) reformulating the cell membrane preparations in a buffer containing pH buffer, inorganic salt, BSA and sucrose, optionally glycerol, such that the total membrane protein concentration is identical or same for said membrane preparations; and 3) depositing said cell membrane preparations onto a substrate surface to form a microarray.

22. A biological, biochemical, or chemical analysis assembly, comprising:
a device and a reagent solution;
said device includes a substrate having a functionalized surface for supporting a plurality of microspots of either biological membranes, membrane-bound proteins, or lipid receptors, arranged in an ordered fashion;
said device is characterized as suited for evaluating certain targets and performing an assay for one of the following purposes: a) determining the relative tissue distribution of a particular target in different tissues or cells; b) determining the abnormal expression level of a particular target in a disease or abnormal tissue or cell; c) determining protein-protein interactions; or d) determining lipid receptor-protein interactions; and
said reagent solution includes either a binder, marker, or a target protein.
